(19) United States
(12) Patent Application Publication
(10) Pub. No.: US 2024/0024161 A1
(43) Pub. Date: Jan. 25, 2024

(54) MEDICAL FACE GUARD, MEDICAL FACE SHIELD, MEDICAL FRAME, AND MEDICAL FACE GUARD MANUFACTURING METHOD

(71) Applicant: SONY GROUP CORPORATION, TOKYO (JP)

(72) Inventor: KAZUYA SAITO, TOKYO (JP)

(21) Appl. No.: 18/254,188

(22) PCT Filed: Nov. 22, 2021
(86) PCT No.: PCT/JP2021/042802

§ 371 Date: May 24, 2023

(30) Foreign Application Priority Data
Dec. 1, 2020 (JP) 2020-199594

(51) Int. Cl. A61F 9/02; A41D 13/05

(52) U.S. Cl. CPC A61F 9/029; A61F 9/022; A41D 13/05

(57) ABSTRACT

A medical face guard (1) includes: a frame (10) to be worn on a head of a medical worker; a polarization shield (20) attached to the frame (10) so as to cover at least a front of eyes of the medical worker and transmitting only light in a predetermined polarization state; and a face shield (30) that is transmissive and has a low birefringence index, the face shield (30) being attached to the frame (10) so as to cover at least the front of the eyes of the medical worker, and positioned in front of the polarization shield (20) in a frontward direction with respect to the eyes.

### FIELD

The present disclosure relates to a medical face guard, a medical face shield, a medical frame, and a medical face guard manufacturing method.

### BACKGROUND

There is a technique, used during surgery, by which a video of a medical imaging device such as an endoscope or a microscope is displayed on a display device as a three-dimensional image so as to provide a practitioner with a video suitable for the surgery. An example of this is a method in which a medical worker such as a practitioner wears glasses having a polarization shield that enables three-dimensional viewing (Patent Literature 1).

### CITATION LIST

### Patent Literature

Patent Literature 1: JP 6747296 B2

### SUMMARY

### Technical Problem

Covering the front of the polarization shield with a transmissive face shield is considered to further improve the protection performance. This, however, has a problem of breakdown of the polarization state by the face shield.

The present disclosure provides, as one aspect, a medical face guard, a medical face shield, a medical frame, and a medical face guard manufacturing method capable of suppressing breakdown of the polarization state.

### Solution to Problem

A medical face guard according to one aspect of the present disclosure includes: a frame to be worn on a head of a medical worker; a polarization shield attached to the frame so as to cover at least a front of eyes of the medical worker and transmitting only light in a predetermined polarization state; and a face shield that is transmissive and has a low birefringence index, the face shield being attached to the frame so as to cover at least the front of the eyes of the medical worker, and positioned in front of the polarization shield in a frontward direction with respect to the eyes.

A medical face shield according to one aspect of the present disclosure is a face shield which is transmissive and has a low birefringence index, the medical face shield being attached to a frame to which a polarization shield that transmits only light in a predetermined polarization state is attached so as to cover at least a front of the eyes of a medical worker, the medical face shield being positioned in front of the polarization shield in a frontward direction with respect to the eyes.

A medical frame according to one aspect of the present disclosure is a medical frame to which a polarization shield that transmits only light in a predetermined polarization state so as to cover at least a front of the eyes of a medical worker and a transmissive face shield having a low birefringence index that is located in front of the polarization shield in a frontward direction with respect to the eyes, are attached.

A medical face guard manufacturing method according to one aspect of the present disclosure includes: a step of preparing a face shield that is transmissive and has a low birefringence index; and a step of attaching a polarization shield that transmits only light in a predetermined polarization state, and the face shield to a frame so as to cover at least a front of eyes of a medical worker such that the face shield is positioned in front of the polarization shield in a frontward direction with respect to the eyes.

### BRIEF DESCRIPTION OF DRAWINGS

FIG. 1 is a diagram illustrating an example of a schematic configuration of a face guard according to an embodiment.

FIG. 2 is a diagram illustrating an example of a schematic configuration of a frame.

FIG. 3 is a diagram illustrating an example of a schematic configuration of a frame.

FIG. 4 is a diagram illustrating an example of a schematic configuration of a frame.

FIG. 5 is a diagram illustrating an example of a schematic configuration of a frame.

FIG. 6 is a diagram illustrating an example of a schematic configuration of a frame.

FIG. 7 is a diagram illustrating an example of a schematic configuration of a polarization shield.

FIG. 8 is a diagram illustrating an example of a schematic configuration of a face shield.

FIG. 9 is a flowchart illustrating an example of a method of manufacturing a face guard.

### DESCRIPTION OF EMBODIMENTS

Embodiments of the present disclosure will be described below in detail with reference to the drawings. Note that, in each of the following embodiments, the same elements are denoted by the same reference symbols, and a repetitive description thereof will be omitted.

The present disclosure will be described in the following order.
1. Embodiments
2. Modification
3. Effects

### 1. EMBODIMENTS

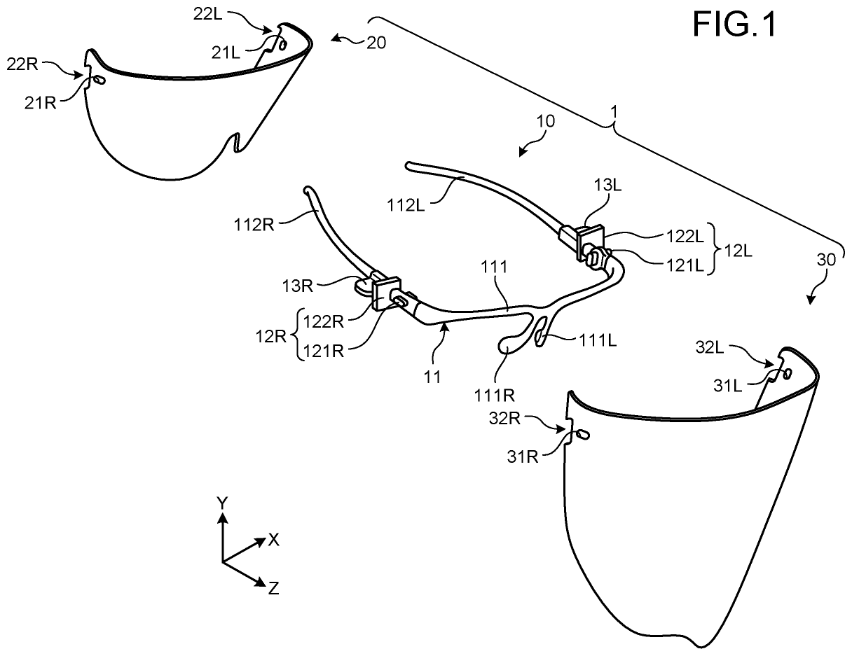

FIG. 1 is an exploded perspective view illustrating an example of a schematic configuration of a face guard according to the embodiment. The face guard is a medical face guard used by a medical worker in a medical field. Examples of the medical field include an operating room and a treatment room. Examples of the medical workers include a practitioner who performs surgery, and a surgical assistant such as an endoscope manipulator and a surgery nurse. FIG. 1 is illustrated with an XYZ coordinate system. The X-axis direction corresponds to the lateral (left-right) direction. The Y-axis direction corresponds to the vertical (up-down) direction. The Z-axis direction corresponds to the depth (front-rear) direction.

The face guard 1 includes a frame 10, a polarization shield 20, and a face shield 30. FIG. 1 illustrates a state in which the frame 10, the polarization shield 20, and the face shield 30 are exploded in Z-axis direction.

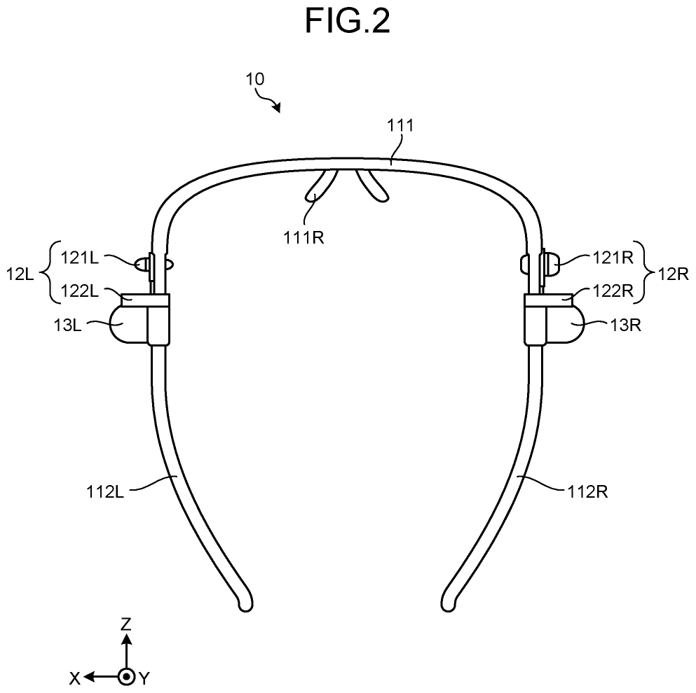

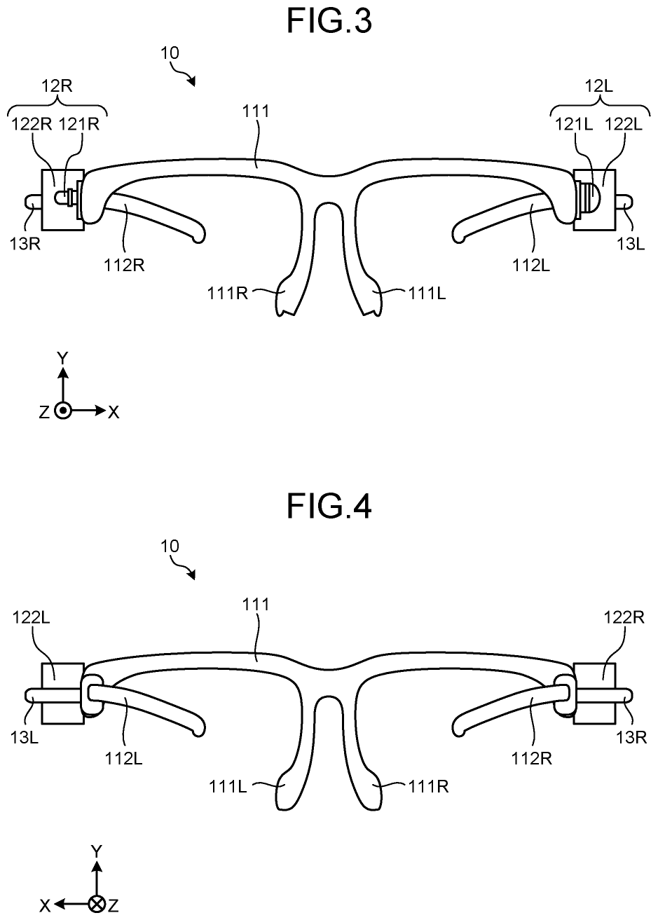

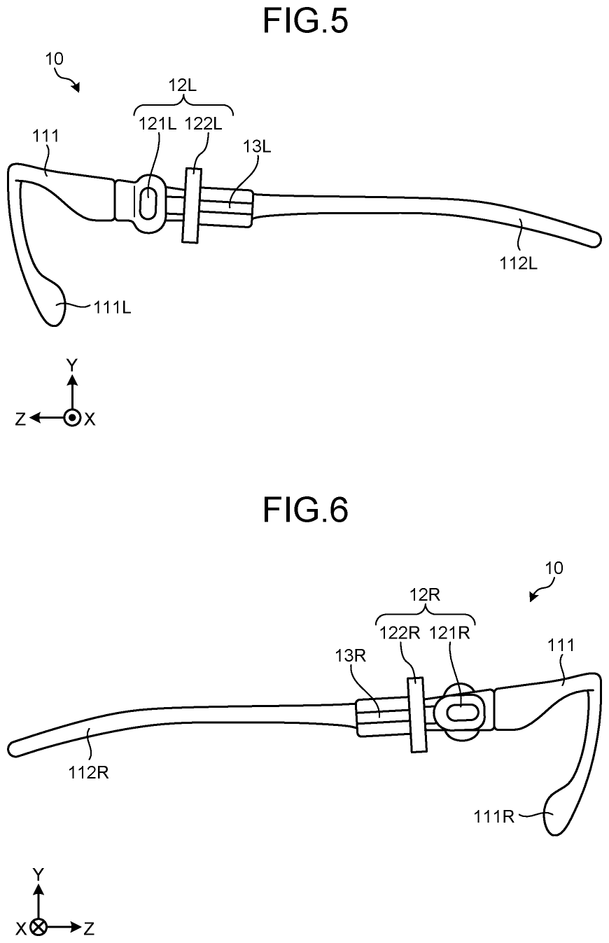

The frame 10 is a medical frame to be worn on the head of the medical worker in a state where the polarization shield and the face shield 30 are attached to the frame 10. The frame 10 will be described with reference to FIGS. 2 to 6. FIGS. 2 to 6 are diagrams illustrating an example of a schematic configuration of a frame. FIG. 2 is a plan view of the frame FIG. 3 is a front view of the frame 10. FIG. 4 is a rear view of the frame 10. FIG. 5 is a left side view of the frame FIG. 6 is a right side view of the frame 10.

The frame 10 is formed using a material resistant to sterilization treatment, for example. Examples of the material of the frame 10 include polycarbonate (PC) and elastomer resin. Alternatively, the surface of the frame 10 may be processed to have resistance to sterilization treatment. With a property enabling sterilization treatment, the frame 10 can be repeatedly used.

The frame 10 includes a frame body 11, an attachment portion 12, and a knob 13. The frame body 11 has a shape that fits the head of the medical worker. In this example, the frame body 11 has an eyeglass shape and extends along the eyes and temple portions of the medical worker. The frame body 11 includes a front portion 111 and a side portion 112.

The front portion 111 extends in the left-right direction (X-axis direction) along eye portions of the medical worker. The front portion 111 includes a pad portion 111L and a pad portion 111R. The pad portion 111L and the pad portion 111R forms a pair of nose pad portions extending downward (Y-axis negative direction) from the central portion of the front portion 111. The pad portion 111L and the pad portion 111R come in contact with the left side portion and the right side portion of the nose of the medical worker respectively so as to position the front portion 111.

The side portion 112 extends in the depth direction (Z-axis direction) along the temple portion of the medical worker. The side portions 112 forms a pair of side portions extending rearward (in the negative direction of the Z-axis) individually from both end portions of the front portion 111. The left side portion 112 is referred to as a side portion 112L in the drawing. The right side portion 112 is referred to as a side portion 112R in the drawing. The side portion 112L and a part of the side portion 112R come in contact with the left ear portion and the right ear portion of the medical worker respectively to position the side portion 112.

The attachment portion 12 is provided to detachably attach the polarization shield 20 and the face shield 30 to the frame 10. The attachment portion 12 is provided to attach the polarization shield 20 and the face shield 30 to the frame body 11 so as to be positioned on opposite sides across the frame 10. In this example, the attachment portion 12 is provided on the side portion 112 of the frame body 11. The attachment portion 12 provided on the side portion 112L is referred to as an attachment portion 12L in the drawing. The attachment portion 12 provided on the side portion 112R is referred to as an attachment portion 12R in the drawing.

The attachment portion 12 includes a fixing member 121 and a fixing member 122. The fixing member 121 and the fixing member 122 of the attachment portion 12L are referred to as a fixing member 121L and a fixing member 122L respectively in the drawings. The fixing member 121 and the fixing member 122 of the attachment portion 12R are referred to as a fixing member 121R and a fixing member 122R, respectively.

The fixing member 121 is a first fixing member that fixes the polarization shield 20 and the face shield 30 from moving in a thickness direction (in this example, the X-axis direction) with respect to the frame body 11. The fixing member 121 can also fix the movement of the polarization shield 20 and the face shield 30 in a plane direction (YZ plane direction in this example) with respect to the frame body 11. The fixing member 121 fixes the polarization shield 20 to the inside of the frame body 11 and fixes the face shield 30 to the outside of the frame body 11.

In this example, the fixing member 121 is a fastener removably (detachably) fixed to the side portion 112 of the frame body 11, and appears as a projection protruding to the inside (head side of the medical worker) and the outside of the frame body 11. The inner projection of the fixing member 121 is fitted to a hole 21 (opening) of the polarization shield 20 to be described below so as to press and fix the polarization shield 20 to the inner surface of the frame body 11. The outer projection of the fixing member 121 is fitted into a hole 31 of the face shield 30 to be described below so as to press and fix the face shield 30 to the outer surface of the frame body 11.

The fixing member 121L and the fixing member 121R may have different shapes. In this case, since the attachment of the polarization shield 20 to the frame 10 is uniquely determined, making it possible, for example, to prevent the polarization shield 20 from being attached with its wrong (front/back) side to the frame 10.

The fixing member 122 is a second fixing member (for example, a rotation stopper) that fixes the polarization shield 20 and the face shield 30 from rotating (rotation about the X axis as a rotation axis in this example) in the plane direction (YZ plane direction in this example) with respect to the frame body 11. In this example, the fixing member 122 is a plate-like member that is engaged with a notch 22 of the polarization shield 20 and a notch 32 of the face shield 30. The fixing member 122 has a plate shape whose plane direction is the XY plane direction, and protrudes to the outside of the frame body 11 behind the fixing member 121 (negative direction on the Z-axis). The fixing member 122 also protrudes to some extent inside the frame body 11. The outer protrusion of the fixing member 122 is engaged with the notch 32 of the face shield 30. The inner protrusion of the fixing member 122 is engaged with the notch 22 of the polarization shield 20.

The knob 13 is gripped when the face guard 1 is worn on the medical worker or when the face guard 1 is removed from the medical worker. For example, when a worker or the like other than the medical worker who uses the face guard 1 grips the knob 13 and attaches the face guard 1 to the medical worker, it is possible to prevent the medical worker from touching the face guard 1. In this example, the knob 13 has a plate shape with the horizontal direction (XZ plane direction) as a plane direction, and protrudes to the outside of the frame body 11 behind the fixing member 122 (in the Z-axis negative direction). The knob 13 located behind the fixing member 122L is referred to as a knob 13L in the drawing. The knob 13 located behind the fixing member 122R is referred to as a knob 13R in the drawing.

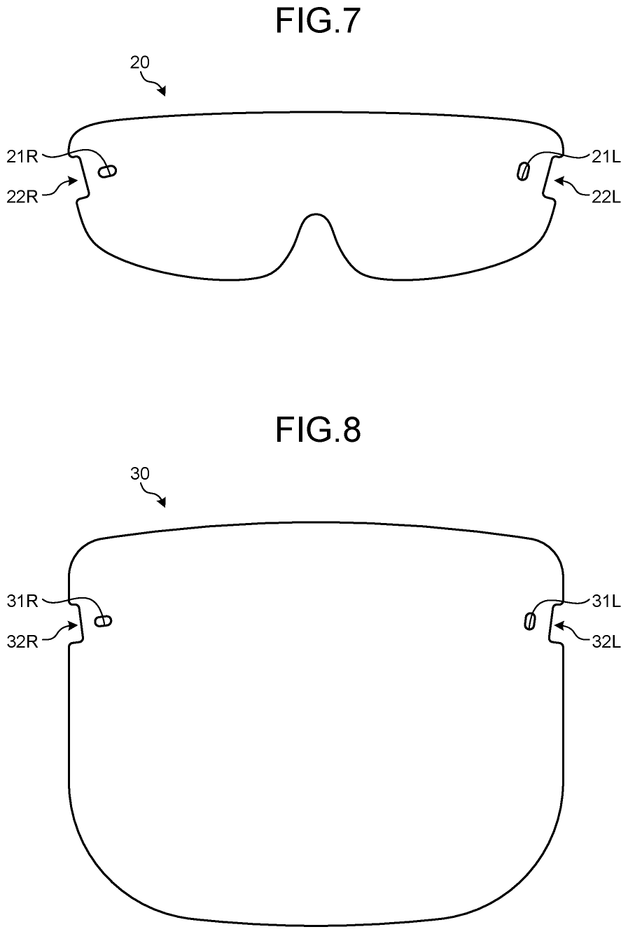

The polarization shield 20 will be described with reference to FIG. 1 and FIG. 7. As illustrated in FIG. 1, the polarization shield 20 is attached, in a state being curved so as to fit the shape of the frame 10, to the inside of the frame 10. FIG. 7 is a view illustrating an example of a schematic configuration of the polarization shield, and is a front view of the polarization shield 20 in a non-curved state.

The polarization shield 20 is attached to the frame 10 so as to cover at least a portion in front of the eyes of the medical worker. The polarization shield 20 may be formed using a transparent material. The polarization shield 20 may have an anti-fog coating on its inner surface (the surface on the head side of the medical worker). The polarization shield 20 may have a water-repellent coating on its outer surface. There may be a gap for improving air permeability between the frame body 11 of the frame 10 and the polarization shield 20.

The polarization shield 20 has a polarization characteristic of transmitting only light in a predetermined polarization state. An example of the light in the predetermined polarization state is light in a state of being polarized so as to be able to display a three-dimensional image. Examples of the material of such a polarization shield 20 include materials such as polycarbonate (PC) and triacetylcellulose (TAC).

The polarization shield 20 may be a three-dimensional eye shield. In this case, the polarization shield 20 transmits only light in a predetermined polarization state so as to enable three-dimensional viewing of a three-dimensional image. For example, a display device displays a three-dimensional image including light polarized in a predetermined direction and light polarized in an opposite direction. The polarization shield 20 transmits light so as to direct the light polarized in a predetermined direction to the left eye of the medical worker and direct the light polarized in the opposite direction to the right eye of the medical worker.

The polarization shield 20 may be a two-dimensional eye shield. In this case, the polarization shield 20 transmits only light in a predetermined polarization state so as to enable two-dimensional viewing of a three-dimensional image. For example, the polarization shield 20 transmits light so as to direct only one of the light polarized in a predetermined direction and the light polarized in the opposite direction, to the eyes of the medical worker. To facilitate distinction of whether the polarization shield 20 is a two-dimensional eye shield or a three-dimensional eye shield, identification marks such as “2D” and “3D” may be attached to the polarization shield 20.

The polarization shield 20 has a hole 21 and a notch 22. In this example, the holes 21 and the notches 22 are provided as a pair of holes 21 and a pair of notches 22. The hole 21 and the notch 22 on the left side are respectively referred to as a hole 21L and a notch 22L in the drawing. The hole 21 and the notch 22 on the right side are respectively referred to as a hole 21R and a notch 22R in the drawings. As described above, the hole 21 is fitted with the inner projection of the fixing member 121 of the frame 10. The hole 21 L has a shape corresponding to the fixing member 121L. The hole 21 R has a shape corresponding to the fixing member 121R. When the fixing member 121L and the fixing member 121R have different shapes, the hole 21L and the hole 21R also have different shapes correspondingly.

As described above, the notch 22 is engaged with the inner protrusion of the fixing member 122 of the frame 10. The notch 22 has a shape corresponding to the fixing member 122. Since the fixing member 122 has a plate shape as described above, the notch 22 in this example has a recessed shape (such as a U-shape with corners in straight angles or a U-shape with round corners) corresponding to the plate shape. The recessed bottom surface comes in contact with the plate surface of the fixing member 122. The recessed side surface comes in contact with the plate side surface of the fixing member 122.

The face shield 30 will be described with reference to FIG. 1 and FIG. 8. As illustrated in FIG. 1, the face shield 30 is attached, in a state being curved so as to fit the shape of the frame 10, to the outside of the frame 10. FIG. 8 is a diagram illustrating an example of a schematic configuration of the face shield 30, and is a front view of the face shield 30 in a non-curved state.

The face shield 30 is a medical face shield attached to the frame 10 so as to cover at least the front of the eyes of the medical worker. The face shield 30 is a transmissive (for example, transparent) face shield and is located in front of the polarization shield 20 in the frontward direction with respect to the eyes (Z-axis positive direction).

The face shield 30 has a length greater than the length of the polarization shield 20 at least in the vertical direction (Y-axis direction). This enables portions of the head of the medical worker not covered with the polarization shield (parts such as forehead, nose, mouth, jaw, for example) to be covered with the face shield 30 as well. The face shield 30 may have a length greater than the length of the polarization shield also in the lateral direction. This makes it possible to cover portions such as the temples and ears of the medical worker with the face shield 30 as well.

With the face shield 30 covering a range wider than the polarization shield 20, for example, it is possible to improve the protective function of the face guard 1, such as preventing splashes from the patient from adhering to the head of the medical worker. Meanwhile, the medical worker views the image (video) from the display device or the like in a three-dimensional view or a two-dimensional view through the face shield 30 and the polarization shield 20. The conventional face shield is formed of a PET material. Therefore, when image light (for example, three-dimensional image light) having a predetermined polarization state output from the display device or the like is transmitted through the face shield, the polarization state of the image light would greatly break down. This can make it difficult for the medical worker to correctly obtain a three-dimensional view or two-dimensional view of the image light from the display device or the like.

In order to suppress the breakdown of the polarization state by the face shield 30 (so as to maintain the polarization state), the present embodiment adopts the face shield 30 having a low birefringence index. In order to achieve continuous transmission of the light in a predetermined polarization state through the polarization shield 20 after transmission through the face shield 30, the face shield 30 has a low birefringence index that maintains the polarization state of the light. Such a low birefringence index is a birefringence index lower than the birefringence index of a polyethylene terephthalate (PET) film or the like, for example.

An example of an indicator of the evaluation of the low birefringence index is a retardation value. The face shield 30 may have a retardation value of 10 nm or less. This enables achievement of appropriate three-dimensional view or two-dimensional view from the viewpoint of practical use.

The material of the face shield 30 may be appropriately selected within a range having a low birefringence index. Specific examples of the material include triacetylcellulose (TAC), acrylic (PMMA), polycarbonate (PC), and cycloolefin polymer (COP).

The dimensions (thickness and the like) of the face shield 30 may be appropriately designed so as to obtain the above-described low birefringence index. The thickness of the face shield 30 is about several tens μm to several hundreds μm, for example. From the viewpoint of securing strength and the like, the face shield 30 may have a thickness of 100 μm or more.

In addition to designing the materials, dimensions, and the like as described above, it is also allowable, in the manufacturing steps, to select (screen) only the face shield 30 having a desired low birefringence index. For example, only the face shield 30 satisfying the specification may be selected by measuring the above-described retardation value. An example of the specification is a specification in which the upper limit value of the retardation value is 10 nm. In consideration of the margin, a retardation value lower than 10 nm to some extent may be set as the upper limit value.

The face shield 30 has a hole 31 and a notch 32. In this example, the holes 31 and the notches 32 are provided as a pair of holes 31 and a pair of notches 32, respectively. The hole 31 and the notch 32 on the left side are respectively referred to as a hole 31L and a notch 32L in the drawing. The hole 31 and the notch 32 on the right side are respectively referred to as a hole 31R and a notch 32R in the drawings.

As described above, hole 31 is fitted to the outer projection of the fixing member 121 of the frame 10. The hole 31 L has a shape corresponding to the fixing member 121L. The hole 31 R has a shape corresponding to the fixing member 121R. When the fixing member 121L and the fixing member 121R have different shapes, the hole 31L and the hole 31R also have different shapes correspondingly.

As described above, the notch 32 is engaged with the outer protrusion of the fixing member 122 of the frame 10. The notch 32 has a shape corresponding to the fixing member 122. Since the fixing member 122 has a plate shape as described above, the notch 32 in this example has a recessed shape corresponding to the plate shape. The recessed bottom surface comes in contact with the plate surface of the fixing member 122. The recessed side surface comes in contact with the plate side surface of the fixing member 122.

By attaching the polarization shield 20 and the face shield 30 described above to the frame 10 via the attachment portion 12, it is possible to obtain the face guard 1 including the polarization shield 20, the frame 10, and the face shield 30 in this order toward the front. According to the face guard 1, since the face shield 30 located in front of the polarization shield 20 has a low birefringence index, the polarization state of the image from the display device or the like is less likely to break down. This makes it possible to achieve accurate three-dimensional viewing or two-dimensional viewing through the polarization shield 20.

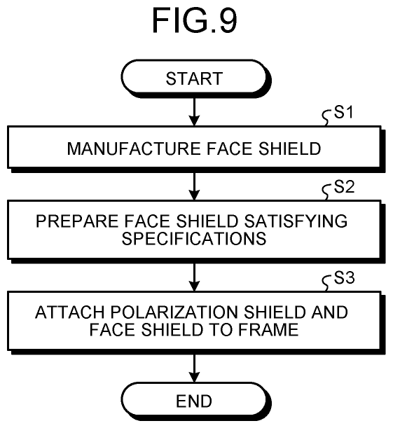

FIG. 9 is a flowchart illustrating an example of a method of manufacturing the face guard.

In step S1, a face shield is manufactured. For example, using TAC as a material, a face shield having a thickness of about several tens μm to one hundred and several tens μm is manufactured. The face shield may be manufactured in plurality.

In step S2, a face shield satisfying specifications is prepared. For example, the retardation value of each of the plurality of face shields manufactured in step S1 described above is measured so as to select a face shield having a measured value of 10 nm or less as the face shield 30.

In step S3, the polarization shield and the face shield are attached to the frame. That is, the face shield 30 prepared in step S2 described above is attached, together with the polarization shield 20, to the frame 10 in a manner as illustrated in FIG. 1 described above to assemble the face guard 1.

For example, the face guard 1 can be manufactured as described above.

### 2. MODIFICATION

Although one embodiment of the present disclosure has been described above, the disclosed technology is not limited to the above embodiment. Some modifications will be described.

The above embodiment has described an example in which the polarization shield 20 and the face shield 30 are located on opposite sides across the frame 10. However, in a range where the polarization shield 20 is located in front of the frame 10, the polarization shield 20 and the face shield 30 may be collectively arranged on the front side of the frame 10 or may be collectively arranged on the rear side of the frame 10. A specific configuration for attaching the polarization shield 20 and the face shield 30 to the frame 10 is not limited to the above-described attachment portion 12. For example, the polarization shield 20 and the face shield 30 may be fixed to the frame 10 by the fixing member 121 alone. The fixing member 121 is not limited to a fastener, and any configuration capable of fixing the polarization shield 20 and the face shield 30 to the frame 10 may be used.

The fixing member 121 and the fixing member 121R of the frame 10 may have the same shape, and the hole 31L and the hole 31R of the face shield 30 may have the same shape. The similar applies to the hole 21L and the hole 21R of the polarization shield 20.

The face guard 1 may have an eyeglass shape as a whole. In this case, parts such as the mouth and jaw of the medical worker may be exposed without being covered with the face shield 30.

The indicator for evaluating the low birefringence index of the face shield 30 may be various indicators other than the retardation value.

### 3. EFFECTS

The embodiment described above is specified as follows, for example. As described with reference to FIG. 1 and the like, the face guard 1 is a medical face guard including: the frame 10 is to be worn on the head of a medical worker; the polarization shield 20 attached to the frame 10 so as to cover at least the front of the eyes of the medical worker and transmitting only light in a predetermined polarization state; and the face shield 30, which is transmissive and has a low birefringence index, attached to the frame 10 so as to cover at least the front of the eyes of the medical worker, positioned in front of the polarization shield 20 in the frontward direction with respect to the eyes (Z-axis positive direction). The polarization shield 20 may be a three-dimensional eye shield that transmits only light in a predetermined polarization state so as to enable three-dimensional viewing of a three-dimensional image. Alternatively, the polarization shield 20 may be a two-dimensional eye shield that transmits only light in a predetermined polarization state so as to enable two-dimensional viewing of a three-dimensional image.

According to the face guard 1, since the face shield 30 located in front of the polarization shield 20 has a low birefringence index, the polarization state of the image from the display device or the like is less likely to break down. This makes it possible to achieve accurate three-dimensional viewing or two-dimensional viewing through the polarization shield 20.

In order to achieve continuous transmission of the light in a predetermined polarization state through the polarization shield 20 after transmission through the face shield 30, the face shield 30 may have a low birefringence index that maintains the polarization state of the light. The face shield 30 having such a low birefringence index, for example, makes it possible to achieve appropriate three-dimensional viewing or two-dimensional viewing.

The face shield 30 may have a retardation value of 10 nm or less. For example, the face shield 30 having such a retardation value makes it possible to achieve appropriate three-dimensional viewing or two-dimensional viewing.

The material of the face shield 30 may contain at least one of triacetylcellulose (TAC), acrylic (PMMA), polycarbonate (PC), or cycloolefin polymer (COP). For example, the face shield 30 can be manufactured using such a material.

The face shield 30 may have a length greater than the length of the polarization shield 20 at least in the vertical direction (Y-axis direction). This allows a portion of the head of the medical worker not covered with the polarization shield to be also covered with the face shield 30, making it possible to improve the protective function of the face guard 1.

The frame 10 may include the frame body 11 and the attachment portion 12 that attaches the polarization shield 20 and the face shield 30 to the frame body 11 so as to be positioned on opposite sides across the frame body 11. This configuration makes it possible dispose the face shield 30 in front of the polarization shield 20.

The attachment portion 12 may include: the fixing member 121 (first fixing member) that fixes the face shield 30 from moving in the thickness direction (X-axis direction) with respect to the frame body 11; and the fixing member 122 (second fixing member) that fixes the face shield 30 from rotating in the surface direction (YZ plane) with respect to the frame body 11. This configuration makes it possible to firmly fix the face shield 30 to be attached to the frame 10.

The face shield 30 may be detachably attached to the frame 10, the fixing member 121 may be a fastener, and the face shield 30 may have the hole 31 to be fitted with the fixing member 121. For example, the face shield 30 can be detachably attached to the frame 10 using such fitting. This makes it possible to use the face shield 30 as a disposable face shield, for example.

The fixing member 122 is a plate-like member, and the face shield 30 may have the notch 32 engaged with the fixing member 122. This makes it possible to prevent the face shield from rotating using such engagement.

The face shield 30 described with reference to FIG. 1 and the like is also one aspect of the present disclosure. That is, the face shield 30 is a medical face shield, which is transmissive and has a low birefringence index, configured to attached to the frame 10 to which the polarization shield 20 that transmits only light in a predetermined polarization state is attached so as to cover at least the front of the eyes of the medical worker, the face shield 30 being positioned in front of the polarization shield 20 in the frontward direction with respect to the eyes. Such a face shield 30 can also make the polarization state less likely to break down as described above.

The frame 10 described with reference to FIG. 1 and the like is also one aspect of the present disclosure. That is, the frame 10 is a medical frame to which the polarization shield 20 that transmits only light in a predetermined polarization state so as to cover at least the front of the eyes of the medical worker and the face shield which is transmissive and has a low birefringence index that is located in front of the polarization shield 20 in the frontward direction with respect to the eyes, are attached. Even with such a frame 10, the polarization state can be made less likely to break down as described above.

The method of manufacturing the face guard 1 described with reference to FIG. 9 and the like is also one aspect of the present disclosure. That is, the manufacturing method includes a step (steps S1 and S2) of preparing the face shield 30 which is transmissive and has a low birefringence index, and a step (step S3) of attaching the polarization shield 20, which transmits only light in a predetermined polarization state, and the face shield 30 to the frame 10 so as to cover at least the front of the eyes of the medical worker such that the face shield 30 is positioned in front of the polarization shield 20 in the frontward direction with respect to the eyes. The face guard 1 described above can be manufactured in this manner, for example.

Note that the effects described in the present disclosure are merely examples and are not limited to the disclosed contents. There may be other effects.

The embodiments of the present disclosure have been described above. However, the technical scope of the present disclosure is not limited to the above-described embodiments, and various modifications can be made without departing from the scope of the present disclosure. Moreover, it is allowable to combine the components across different embodiments and modifications as appropriate.

Note that the present technique can also have the following configurations.
(1) A medical face guard comprising:
   a frame to be worn on a head of a medical worker;
   a polarization shield attached to the frame so as to cover at least a front of eyes of the medical worker and transmitting only light in a predetermined polarization state; and
   a face shield that is transmissive and has a low birefringence index, the face shield being attached to the frame so as to cover at least the front of the eyes of the medical worker, and positioned in front of the polarization shield in a frontward direction with respect to the eyes.
(2) The medical face guard according to (1),
   wherein the face shield has a low birefringence index that maintains the polarization state of the light in order to achieve continuous transmission of the light in a predetermined polarization state through the polarization shield after transmission through the face shield.
(3) The medical face guard according to (1) or (2),
   wherein the face shield has a retardation value of 10 nm or less.
(4) The medical face guard according to any one of (1) to (3),
   wherein a material of the face shield contains at least one of triacetylcellulose, acrylic, polycarbonate, or cycloolefin polymer.
(5) The medical face guard according to any one of (1) to (4),
   wherein the face shield has a length greater than a length of the polarization shield at least in a vertical direction.
(6) The medical face guard according to any one of (1) to (5),
   wherein the frame includes:
   a frame body; and
   an attachment portion that attaches the polarization shield and the face shield to the frame body such that the polarization shield and the face shield are positioned on opposite sides across the frame body.
(7) The medical face guard according to (6),
   wherein the attachment portion includes:
   a first fixing member that fixes the face shield from moving in a thickness direction with respect to the frame body; and
   a second fixing member that fixes the face shield from rotating in a plane direction with respect to the frame body.
(8) The medical face guard according to (7),
   wherein the face shield is detachably attached to the frame,
   the first fixing member is a fastener, and
   the face shield has a hole to be fitted with the first fixing member.
(9) The medical face guard according to (7) or (8),
   wherein the second fixing member is a plate-like member, and
   the face shield has a notch to be engaged with the second fixing member.
(10) The medical face guard according to any one of (1) to (9),
   wherein the polarization shield is a three-dimensional eye shield that transmits only light in the predetermined polarization state so as to enable three-dimensional viewing of a three-dimensional image.
(11) The medical face guard according to any one of (1) to (9),
   wherein the polarization shield is a two-dimensional eye shield that transmits only light in the predetermined polarization state so as to enable two-dimensional viewing of a three-dimensional image.
(12) A medical face shield which is transmissive and has a low birefringence index, the medical face shield being attached to a frame to which a polarization shield that transmits only light in a predetermined polarization state is attached so as to cover at least a front of the eyes of a medical worker, the medical face shield being positioned in front of the polarization shield in a frontward direction with respect to the eyes.
(13) A medical frame to which a polarization shield that transmits only light in a predetermined polarization state so as to cover at least a front of the eyes of a medical worker and a transmissive face shield having a low birefringence index that is located in front of the polarization shield in a frontward direction with respect to the eyes, are attached.
(14) A medical face guard manufacturing method comprising:
   a step of preparing a face shield that is transmissive and has a low birefringence index; and
   a step of attaching a polarization shield that transmits only light in a predetermined polarization state, and the face shield to a frame so as to cover at least a front of eyes of a medical worker such that the face shield is positioned in front of the polarization shield in a frontward direction with respect to the eyes.

### REFERENCE SIGNS LIST

1 FACE GUARD
10 FRAME
12 ATTACHMENT PORTION
13 KNOB
20 POLARIZATION SHIELD
21 HOLE
22 NOTCH
30 FACE SHIELD
31 HOLE
32 NOTCH
111 FRONT PORTION
112 SIDE PORTION
121 FIXING MEMBER (FIRST FIXING MEMBER)
122 FIXING MEMBER (SECOND FIXING MEMBER)

1. A medical face guard comprising:
a frame to be worn on a head of a medical worker;
a polarization shield attached to the frame so as to cover at least a front of eyes of the medical worker and transmitting only light in a predetermined polarization state; and
a face shield that is transmissive and has a low birefringence index, the face shield being attached to the frame so as to cover at least the front of the eyes of the medical worker, and positioned in front of the polarization shield in a frontward direction with respect to the eyes.

2. The medical face guard according to claim 1,
wherein the face shield has a low birefringence index that maintains the polarization state of the light in order to achieve continuous transmission of the light in a predetermined polarization state through the polarization shield after transmission through the face shield.

3. The medical face guard according to claim 1,
wherein the face shield has a retardation value of 10 nm or less.

4. The medical face guard according to claim 1,
wherein a material of the face shield contains at least one of triacetylcellulose, acrylic, polycarbonate, or cycloolefin polymer.

5. The medical face guard according to claim 1,
wherein the face shield has a length greater than a length of the polarization shield at least in a vertical direction.

6. The medical face guard according to claim 1,
wherein the frame includes:
a frame body; and
an attachment portion that attaches the polarization shield and the face shield to the frame body such that the polarization shield and the face shield are positioned on opposite sides across the frame body.

7. The medical face guard according to claim 6,
wherein the attachment portion includes:
a first fixing member that fixes the face shield from moving in a thickness direction with respect to the frame body; and
a second fixing member that fixes the face shield from rotating in a plane direction with respect to the frame body.

8. The medical face guard according to claim 7,
wherein the face shield is detachably attached to the frame,
the first fixing member is a fastener, and
the face shield has a hole to be fitted with the first fixing member.

9. The medical face guard according to claim 7,
wherein the second fixing member is a plate-like member, and
the face shield has a notch to be engaged with the second fixing member.

10. The medical face guard according to claim 1,
wherein the polarization shield is a three-dimensional eye shield that transmits only light in the predetermined polarization state so as to enable three-dimensional viewing of a three-dimensional image.

11. The medical face guard according to claim 1,
wherein the polarization shield is a two-dimensional eye shield that transmits only light in the predetermined polarization state so as to enable two-dimensional viewing of a three-dimensional image.

12. A medical face shield which is transmissive and has a low birefringence index, the medical face shield being attached to a frame to which a polarization shield that transmits only light in a predetermined polarization state is attached so as to cover at least a front of the eyes of a medical worker, the medical face shield being positioned in front of the polarization shield in a frontward direction with respect to the eyes.

13. A medical frame to which a polarization shield that transmits only light in a predetermined polarization state so as to cover at least a front of the eyes of a medical worker and a transmissive face shield having a low birefringence index that is located in front of the polarization shield in a frontward direction with respect to the eyes, are attached.

14. A medical face guard manufacturing method comprising:
a step of preparing a face shield that is transmissive and has a low birefringence index; and
a step of attaching a polarization shield that transmits only light in a predetermined polarization state, and the face shield to a frame so as to cover at least a front of eyes of a medical worker such that the face shield is positioned in front of the polarization shield in a frontward direction with respect to the eyes.
